(19) United States
(12) Patent Application Publication
(10) Pub. No.: US 2008/0160077 A1
(43) Pub. Date: Jul. 3, 2008

(54) Soft Gel Formulations

(75) Inventor: Henryk Borowy-Borowski, Ottawa (CA)

Correspondence Address: MORGAN, LEWIS & BOCKIUS LLP (SF), One Market, Spear Street Tower, Suite 2800, San Francisco, CA, 94105, US

(73) Assignee: Zymes, LLC, Hasbrouck Heights, NJ (US)

(21) Appl. No.: 11/946,020

(22) Filed: Nov. 27, 2007

Related U.S. Application Data
(60) Provisional application No. 60/867,389, filed on Nov. 27, 2006.
(60) Provisional application No. 60/886,212, filed on Jan. 23, 2007.

(51) Int. Cl. A61K 9/48; A61K 47/14; A61K 38/43

(52) U.S. Cl. 424/456; 514/785; 424/94.1; 514/787

(57) ABSTRACT

This invention relates to an improved formulation methodology for bioactive lipophilic molecules, such as Coenzyme Q₁₀ (CoQ₁₀) and its reduced analogs (ubiquinols). Further provided are methods of producing soft gel capsules of this formulation.

### CROSS-REFERENCE TO RELATED APPLICATIONS

This application claims priority to U.S. Provisional Patent Applications 60/867,389 filed on Nov. 27, 2006 and 60/886,212 filed on Jan. 23, 2007, which are herein incorporated by reference in its entirety for all purposes.

### BACKGROUND OF THE INVENTION

It is desirable to produce pharmaceutical or nutraceutical or cosmetic formulations which are smaller and/or lighter, as these formulations are easier for patients to consume, thus increasing patient compliance with therapeutic regimens. This, and other advantages, are provided by the current invention.

### SUMMARY OF THE INVENTION

The present invention provides smaller and/or lighter pharmaceutical and/or nutraceutical and/or cosmeceutical formulations of bioactive lipophilic molecules. The novel formulations can be characterized by improved bioavailability of the bioactive lipophilic molecule, providing opportunities for products with reduced total amounts of bioactive lipophilic molecules, increased bioavailability, reduced formulation and/or capsule sizes and associated cost. The invention further provides methods of producing the novel formulations. Preferred applications for the formulations lie in soft gel capsules. A preferred soft gel formulation of the invention includes ubiquinone/ubiquinol, a surfactant, such as polyoxyethanyl-a-tocopheryl-sebacate (PTS), a viscosity enhancer (e.g., bees wax), as well as a lipophilic carrier, such as rice bran oil or fish oil. Another preferred soft gel formulation of the invention includes CoQ₁₀, a surfactant, such as polyoxyethanyl-a-tocopheryl-sebacate (PTS), a viscosity enhancer (e.g., bees wax), as well as a lipophilic carrier, such as rice bran oil or fish oil. Preferred formulations of the invention contain a minimal amount of viscosity enhancer and surfactant.

It is an object of the present invention to provide a soft gel formulation of a ubiquinone/ubiquinol (such as CoQ₁₀) with significantly greater bioavailability than existing soft or dry formulations. The invention further provides a soft gel formulation of ubiquinone/ubiquinol (such as CoQ₁₀) and methodology of administration that produces greater absorption of the ubiquinone/ubiquinol (such as CoQ₁₀) into the intestine. Another object is to minimize the ingested volume required to maintain a given ubiquinone/ubiquinol (such as CoQ₁₀) blood content. Another object is to provide a process that keeps ubiquinone/ubiquinol (such as CoQ₁₀) in solution in readily absorbed materials, that themselves have beneficial effects.

These and other aspects and advantages of the present invention will become apparent to those skilled in the art after considering the following detailed description of the invention.

### BRIEF DESCRIPTION OF THE FIGURES

FIG. 1A shows a representative particle size-distribution curves for a 16-fold dilution of PTS in water.

FIG. 1B shows a representative particle size-distribution curve for a 16-fold dilution of a 3:1 PTS/CoQ₁₀ composition in water.

### DETAILED DESCRIPTION OF THE INVENTION

### I. Definitions

The term “monoterpene” as used herein, refers to a compound having a 10-carbon skeleton with non-linear branches. A monoterpene refers to a compound with two isoprene units connected in a head-to-end manner. The term “monoterpene” is also intended to include “monoterpenoid”, which refers to a monoterpene-like substance and may be used loosely herein to refer collectively to monoterpenoid derivatives as well as monoterpenoid analogs. Monoterpenoids can therefore include monoterpenes, alcohols, ketones, aldehydes, ethers, acids, hydrocarbons without an oxygen functional group, and so forth.

As used herein, the term “phospholipid” is recognized in the art, and refers to phosphatidyl glycerol, phosphatidyl inositol, phosphatidyl serine, phosphatidyl choline, phosphatidyl ethanolamine, as well as phosphatidic acids, ceramides, cerebrosides, sphingomyelins and cardiolipins.

As used herein, the term “antioxidant” is recognized in the art and refers to synthetic or natural substances that prevent or delay the oxidative deterioration of a compound. Exemplary antioxidants include tocopherols, flavonoids, catechins, superoxide dismutase, lecithin, gamma oryzanol; vitamins, such as vitamins A, C (ascorbic acid) and E and beta-carotene; natural components such as camosol, carnosic acid and rosmanol found in rosemary and hawthorn extract, proanthocyanidins such as those found in grapeseed or pine bark extract, and green tea extract.

The term “flavonoid” as used herein is recognized in the art and is intended to include those plant pigments found in many foods that are thought to help protect the body from cancer. These include, for example, epi-gallo catechin gallate (EGCG), epi-gallo catechin (EGC) and epi-catechin (EC).

The term “alkyl,” by itself or as part of another substituent, means, unless otherwise stated, a straight or branched chain, or cyclic hydrocarbon radical, or combination thereof, which may be fully saturated, mono- or polyunsaturated and can include di- and multi-valent radicals, having the number of carbon atoms designated (i.e. C₁-C₁₀ means one to ten carbons). Examples of saturated hydrocarbon radicals include groups such as methyl, ethyl, n-propyl, isopropyl, n-butyl, t-butyl, isobutyl, sec-butyl, cyclohexyl, (cyclohexyl)ethyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “alkyl,” unless otherwise noted, is also meant to include those derivatives of alkyl defined in more detail below as “heteroalkyl,” “cycloalkyl” and “alkylene.” The term “alkylene” by itself or as part of another substituent means a divalent radical derived from an alkane, as exemplified by —CH₂CH₂CH₂CH₂—. Typically, an alkyl group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms.

The terms “alkoxy,” “alkylamino” and “alkylthio” refer to those groups having an alkyl group attached to the remainder of the molecule through an oxygen, nitrogen or sulfur atom, respectively. Similarly, the term “dialkylamino” is used in a conventional sense to refer to —NR′R″ wherein the R groups can be the same or different alkyl groups.

The term “acyl” or “alkanoyl” by itself or in combination with another term, means, unless otherwise stated, a stable straight or branched chain, or cyclic hydrocarbon radical, or combinations thereof, consisting of the stated number of carbon atoms and an acyl radical on at least one terminus of the alkane radical.

The term “heteroalkyl,” by itself or in combination with another term, means, unless otherwise stated, a stable straight or branched chain, or cyclic hydrocarbon radical, or combinations thereof, consisting of the stated number of carbon atoms and from one to three heteroatoms selected from the group consisting of O, N, Si and S, and wherein the nitrogen and sulfur atoms may optionally be oxidized and the nitrogen heteroatom may optionally be quaternized. The heteroatom(s) O, N and S may be placed at any interior position of the heteroalkyl group. The heteroatom Si may be placed at any position of the heteroalkyl group, including the position at which the alkyl group is attached to the remainder of the molecule. Examples include —CH₂—CH₂—O—CH₃, —CH₂—CH₂—NH—CH₃, —CH₂—CH₂—N(CH₃)—CH₃, —CH₂—S—CH₂—CH₃, —CH₂—CH₂—S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. Up to two heteroatoms may be consecutive, such as, for example, —CH₂—NH—OCH₃ and —CH₂—O—Si(CH₃)₃. Also included in the term “heteroalkyl” are those radicals described in more detail below as “heteroalkylene” and “heterocycloalkyl.” The term “heteroalkylene” by itself or as part of another substituent means a divalent radical derived from heteroalkyl, as exemplified by —CH₂—CH₂—S—CH₂CH₂— and —CH₂—S—CH₂—CH₂—NH—CH₂—. For heteroalkylene groups, heteroatoms can also occupy either or both of the chain termini. Still further, for alkylene and heteroalkylene linking groups, no orientation of the linking group is implied.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include cyclopentyl, cyclohexyl, 1-cyclohexenyl, 3-cyclohexenyl, cycloheptyl, and the like. Examples of heterocycloalkyl include 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like.

The terms “halo” or “halogen,” by themselves or as part of another substituent, mean, unless otherwise stated, a fluorine, chlorine, bromine, or iodine atom. Additionally, terms such as “fluoroalkyl,” are meant to include monofluoroalkyl and polyfluoroalkyl.

The term “aryl,” employed alone or in combination with other terms (e.g., aryloxy, arylthioxy, arylalkyl) means, unless otherwise stated, an aromatic substituent which can be a single ring or multiple rings (up to three rings), which are fused together or linked covalently. “Heteroaryl” are those aryl groups having at least one heteroatom ring member. Typically, the rings each contain from zero to four heteroatoms selected from N, O, and S, wherein the nitrogen and sulfur atoms are optionally oxidized, and the nitrogen atom(s) are optionally quaternized. The “heteroaryl” groups can be attached to the remainder of the molecule through a heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl ring systems are selected from the group of acceptable substituents described below. The term “arylalkyl” is meant to include those radicals in which an aryl group is attached to an alkyl group (e.g., benzyl, phenethyl, pyridylmethyl and the like) or a heteroalkyl group (e.g., phenoxymethyl, 2-pyridyloxymethyl, 3-(1-naphthyloxy)propyl, and the like).

Each of the above terms (e.g., “alkyl,” “heteroalkyl” and “aryl”) are meant to include both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be a variety of groups selected from, for example: —OR′, ═O, ═NR′, ═N—OR′, —NR′R″, —SR′, -halogen, —SiR′R″R′″, —OC(O)R′, —C(O)R′, —CO₂R′, CONR′R″, —OC(O)NR′R″, —NR″C(O)R′, —NR′, —C(O)NR″R′″, —NR″C(O)₂R′, —NH—C(NH₂)═NH, —NR′C(NH₂)═NH, —NH—C(NH₂)═NR′, —S(O)R′, —S(O)₂R′, —S(O)₂NR′R″, —CN and —NO₂ in a number ranging from zero to (2N+1), where N is the total number of carbon atoms in such radical. R′, R″ and R′″ each independently refer to hydrogen, unsubstituted (C₁-C₈)alkyl and heteroalkyl, unsubstituted aryl, aryl substituted with 1-3 halogens, unsubstituted alkyl, alkoxy or thioalkoxy groups, or aryl-(C₁-C₄)alkyl groups. When R′ and R″ are attached to the same nitrogen atom, they can be combined with the nitrogen atom to form a 5-, 6-, or 7-membered ring. For example, —NR′R″ is meant to include 1-pyrrolidinyl and 4-morpholinyl. From the above discussion of substituents, one of skill in the art will understand that the term “alkyl” is meant to include groups such as haloalkyl (e.g., —CF₃ and —CH₂CF₃) and acyl (e.g., —C(O)CH₃, —C(O)CF₃, —C(O)CH₂OCH₃, and the like).

Similarly, substituents for the aryl groups are varied and are selected from: -halogen,—OR′, —OC(O)R′, —NR′R″, —SR′, —R′, —CN, —NO₂, —CO₂R′, —CONR′R″, —C(O)R′, —OC(O)NR′R″, —NR″C(O)R′, —NR″C(O)₂R′, —NR′C(O)NR″R′″, —NH—C(NH₂)═NH, —NR′C(NH₂)═NH, —NH—C(NH₂)═NR′, —S(O)R′, —S(O)₂R′, —S(O)₂NR′R″, —N₃, —CH(Ph)₂, perfluoro(C₁-C₄)alkoxy, and perfluoro(C₁-C₄)alkyl, in a number ranging from zero to the total number of open valences on the aromatic ring system; and where R′, R″ and R′″ are independently selected from hydrogen, (C₁-C₈)alkyl and heteroalkyl, unsubstituted aryl, (unsubstituted aryl)-(C₁-C₄)alkyl, (unsubstituted aryl)oxy-(C₁-C₄)alkyl and perfluoro(C₁-C₄)alkyl.

Two of the substituents on adjacent atoms of the aryl ring may optionally be replaced with a substituent of the formula -T-C(O)—(CH₂)_{q}—U—, wherein T and U are independently —NH—, —O—, —CH₂— or a single bond, and the subscript q is an integer of from 0 to 2. Alternatively, two of the substituents on adjacent atoms of the aryl ring may optionally be replaced with a substituent of the formula -A-(CH₂)ᵣ—B—, wherein A and B are independently —CH₂—, —O—, —NH—, —S—, —S(O)—, —S(O)₂—, —S(O)₂NR′— or a single bond, and r is an integer of from 1 to 3. One of the single bonds of the new ring so formed may optionally be replaced with a double bond. Alternatively, two of the substituents on adjacent atoms of the aryl ring may optionally be replaced with a substituent of the formula —(CH₂)ₛ—X—(CH₂)ₜ—, where s and t are independently integers of from 0 to 3, and X is —O—, —NR′—, —S—, —S(O)—, —S(O)₂—, or —S(O)₂NR′—. The substituent R′ in —NR′— and —S(O)₂NR′— is selected from hydrogen or unsubstituted (C₁-C₆)alkyl.

As used herein, the term “heteroatom” is meant to include, for example, oxygen (O), nitrogen (N), sulfur (S) and silicon (Si).

Certain compounds of the present invention possess asymmetric carbon atoms (optical centers) or double bonds; the racemates, diastereomers, geometric isomers and individual isomers are all encompassed within the scope of the present invention.

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present invention, whether radioactive or not, are intended to be encompassed within the scope of the present invention.

As used herein, the term “leaving group” refers to a portion of a substrate that is cleaved from the substrate in a reaction. The leaving group is an atom (or a group of atoms) that is displaced as stable species taking with it the bonding electrons. Typically the leaving group is an anion (e.g., Cl⁻) or a neutral molecule (e.g., H₂O). Exemplary leaving groups include a halogen, OC(O)R⁶⁵, OP(O)R⁶⁵R⁶⁶, OS(O)R⁶⁵, and OSO₂R⁶⁵. R⁶⁵ and R⁶⁶ are members independently selected from substituted or unsubstituted alkyl, substituted or unsubstituted aryl, substituted or unsubstituted heteroaryl and substituted or unsubstituted heterocycloalkyl. Useful leaving groups include, but are not limited to, other halides, sulfonic esters, oxonium ions, alkyl perchlorates, sulfonates, e.g., arylsulfonates, ammonioalkanesulfonate esters, and alkylfluorosulfonates, phosphates, carboxylic acid esters, carbonates, ethers, and fluorinated compounds (e.g., triflates, nonaflates, tresylates), S R⁶⁵, (R⁶⁵)₃P⁺, (R⁶⁵)₂S⁺, P(O)N(R⁶⁵)₂(R₆₅)₂, p(O)XR⁶⁵X′R⁶⁵ in which each R⁶⁵ is independently selected from the members provided in this paragraph and X and X′ are S or O. The choice of these and other leaving groups appropriate for a particular set of reaction conditions is within the abilities of those of skill in the art (see, for example, March J, ADVANCED ORGANIC CHEMISTRY, 2nd Edition, John Wiley and Sons, 1992; Sandler S R, Karo W, ORGANIC FUNCTIONAL GROUP PREPARATIONS, 2nd Edition, Academic Press, Inc., 1983; and Wade L G, COMPENDIUM OF ORGANIC SYNTHETIC METHODS, John Wiley and Sons, 1980).

“Protecting group,” as used herein refers to a portion of a substrate that is substantially stable under a particular reaction condition, but which is cleaved from the substrate under a different reaction condition. A protecting group can also be selected such that it participates in the direct oxidation of the aromatic ring component of the compounds of the invention. For examples of useful protecting groups, see, for example, Greene et al., PROTECTIVE GROUPS IN ORGANIC SYNTHESIS, 3rd ed., John Wiley & Sons, New York, 1999.

“Ring” as used herein means a substituted or unsubstituted cycloalkyl, substituted or unsubstituted heterocycloalkyl, substituted or unsubstituted aryl, or substituted or unsubstituted heteroaryl. A ring includes fused ring moieties. The number of atoms in a ring is typically defined by the number of members in the ring. For example, a “5- to 7-membered ring” means there are 5 to 7 atoms in the encircling arrangement. The ring optionally included a heteroatom. Thus, the term “5- to 7-membered ring” includes, for example pyridinyl and piperidinyl. The term “ring” further includes a ring system comprising more than one “ring”, wherein each “ring” is independently defined as above.

### II. Introduction

The current invention provides novel formulations for bioactive lipophilic molecules. In some embodiments, there is a first formulation which includes (a) bioactive lipophilic molecule; (b) a surfactant; (c) a lipophilic carrier; and (d) a viscosity enhancer. In some embodiments, there is a second formulation, which comprises the first formulation as well as a pharmaceutically acceptable, nutraceutically acceptable or cosmetically acceptable carrier described herein, such as a tablet, troche, lozenge, capsule (hard, soft or soft gel), syrup or elixir. The term ‘formulation’, when used without the terms ‘first’ or ‘second’ preceding it, can refer to either a first formulation and/or a second formulation. In an exemplary embodiment, the invention provides a second formulation which comprises a first formulation described herein, which is encompassed within a soft gel capsule.

When added to an aqueous solution, the formulations allow for the formation of micelles, wherein particle size of the micelles is surprisingly small, enabling greater bioavailability. An added advantage of the current formulations is their greater health benefits due to the presence of essential polyunsaturated fatty acids, such as omega-3 fatty acids. In addition, the current formulations are stable while containing a smaller amount of viscosity enhancers (e.g., bees wax) and/or surfactants, relative to the active ingredient, than known formulations. Another advantage of the current formulations is their stability with respect to precipitation of one or more components and the active ingredient in particular. In an exemplary embodiment, CoQ₁₀ formulations of the invention are stable under an inert atmosphere or within a soft gel capsule (without visible precipitation) at room temperature for extended amounts of time, such as from about 2 months or more. A further advantage of the invention is the reduction in the size and/or mass of the formulation.

### III. First Formulation

In one aspect, the invention provides a first formulation which comprises: (a) bioactive lipophilic molecule; (b) a surfactant; (c) a lipophilic carrier; and (d) a viscosity enhancer.

Typically, the first formulation includes from about 10% to about 30% (w/w) bioactive lipophilic molecule, from about 15% to about 40% (w/w) surfactant, from about 30% to about 60% (w/w) lipophilic carrier and from about 1% to about 10% (w/w) viscosity enhancer. In an exemplary embodiment, the first formulation includes from about 10% to about 30% (w/w) bioactive lipophilic molecule, from about 15% to about 40% (w/w) of a surfactant which is a member selected from polyoxyethanyl-tocopherol-sebacate (PTS), polyoxyethanyl-sitosterol-sebacate (PSS), polyoxyethanyl-cholesterol-sebacate (PC S), polyoxyethanyl-ubiquinol-sebacate (PQS) and combinations thereof, from about 30% to about 60% (w/w) lipophilic carrier and from about 1% to about 10% (w/w) viscosity enhancer. In an exemplary embodiment, the first formulation includes from about 10% to about 30% (w/w) bioactive lipophilic molecule, from about 15% to about 40% (w/w) of a surfactant, from about 30% to about 60% (w/w) fish oil and from about 1% to about 10% (w/w) viscosity enhancer. In an exemplary embodiment, the first formulation includes from about 10% to about 30% (w/w) bioactive lipophilic molecule, from about 15% to about 40% (w/w) of a surfactant, from about 30% to about 60% (w/w) lipophilic carrier and from about 1% to about 10% (w/w) beeswax. In an exemplary embodiment, the soft gel capsule of the invention includes CoQ₁₀, beeswax, a lipophilic carrier (e.g., fish oil) enriched with omega fatty acid, and a surfactant which is a member selected from polyoxyethanyl-tocopherol-sebacate (PTS), polyoxyethanyl-sitosterol-sebacate (PSS), polyoxyethanyl-cholesterol-sebacate (PCS), polyoxyethanyl-ubiquinol-sebacate (PQS) and combinations thereof.

In another exemplary embodiment, the invention provides a first formulation comprising a) bioactive lipophilic molecule; b) surfactant; c) lipophilic carrier and d) a viscosity enhancer. In this formulation the ratio of the bioactive lipophilic molecule to surfactant is from about 1:1 to about 1:5 (w/w), preferably from about 1:1 to about 1:3 (w/w), more preferably from about 1:1 to about 1:2 (w/w), and more preferably from about 1:1 to about 1:1.5 (w/w), and more preferably about 1.5 (w/w). In this formulation the ratio of bioactive lipophilic molecule to lipophilic carrier is from about 1:1 to about 1:10 (w/w), preferably from about 1:1 to about 1:6 (w/w), and more preferably from about 1:1 to about 1:4 (w/w) and more preferably from about 1:1 to about 1:3 (w/w), and more preferably about 1:3 (w/w). In this formulation the ratio of bioactive lipophilic molecule to viscosity enhancer is from about 1:0.1 to about 1:1 (w/w), preferably from about 1:0.2 to about 1:0.75 (w/w), and more preferably from about 1:0.33 to about 1:0.66 (w/w) and more preferably about 1:0.5 (w/w). In an exemplary embodiment, the ratio of bioactive lipophilic molecule to surfactant is about 1:1.5 (w/w), the ratio of bioactive lipophilic molecule to lipophilic carrier is from about 1:2 to about 1:3 (w/w), and the ratio of bioactive lipophilic molecule to viscosity enhancer is about 1:0.25 to about 1:0.5 (w/w).

### III. (a) Bioactive Lipophilic Molecule

In an exemplary embodiment, the bioactive lipophilic molecule is a member selected from ubiquinone, ubiquinol, asiatic acid, ursolic acid, lutein, astaxanthin, curcumins, beta-carotene, lycopene, resveratrol, lecithin, L-carnitine (or acetyl derivative), tocotrienols, alpha-lipoic acid, salmon oil, grape seed extract, bilberry extract, flaxseed oil, garlic oil, ginkgo biloba extract, pumpkin seed oil, green tea catechins extract, kava, evening primrose oil, wheat germ oil, hyaluronic acid, saw palmetto berry oil extract, ginseng, Japanese knotwood extract, phytosterols, hawthorne, St. John's wort, melatonin, valerian, yohimbe, ephedra, red clover, cayenne, echinacea, arnica Montana, docosahexaenoic acid, analogs thereof (such as ester derivatives and/or amide derivatives) and combinations thereof. Bioactive lipophilic molecule analogs can include any bioactive lipophilic molecule described herein which has had at least one free OH or COOH group converted into an ester. Bioactive lipophilic molecules analogs can include any bioactive lipophilic molecule described herein which has had at least one free NH group converted into an amide. When added to an aqueous solution, the formulations allow for the formation of micelles, wherein particle size of the micelles is surprisingly small, enabling greater bioavailability. An added advantage of the current formulations is their greater health benefits due to the presence of essential polyunsaturated fatty acids, such as omega-3 fatty acids. In addition, the current formulations are stable if they contain a viscosity enhancer (e.g., bees wax) and/or surfactants, relative to the bioactive lipophilic molecule, than known formulations. Another advantage of the current formulations is their stability with respect to precipitation of one or more components and the bioactive lipophilic molecule in particular. In an exemplary embodiment, bioactive lipophilic molecule formulations of the invention are stable under an inert atmosphere or within a soft gel capsule at room temperature for extended amounts of time, such as from about 2 months or more. These formulations can be produced according to a method described herein.

In one aspect, the invention provides a formulation which comprises: (a) a bioactive lipophilic molecule which is a member selected from a ubiquinone, ubiquinol, asiatic acid, ursolic acid, lutein, astaxanthin, curcumins, beta-carotene, lycopene, resveratrol, lecithin, L-carnitine (or acetyl derivative), tocotrienols, alpha-lipoic acid, salmon oil, grape seed extract, bilberry extract, flaxseed oil, garlic oil, ginkgo biloba extract, pumpkin seed oil, green tea catechins extract, kava, evening primrose oil, wheat germ oil, hyaluronic acid, saw palmetto berry oil extract, ginseng, Japanese knotwood extract, phytosterols, hawthorne, St. John's wort, melatonin, valerian, yohimbe, ephedra, red clover, cayenne, echinacea, arnica Montana, docosahexaenoic acid, analogs thereof (such as ester derivatives and/or amide derivatives) and combinations thereof, (b) a surfactant described herein; and a (c) a lipophilic carrier described herein. In an exemplary embodiment, this formulation further comprises (d) a viscosity enhancer described herein.

In an exemplary embodiment, the ubiquinone/ubiquinol of the current invention has a structure which is described herein. In another exemplary embodiment, the ubiquinone/ubiquinol of the current invention has a structure which is a member selected from Formula (I) and Formula (II):

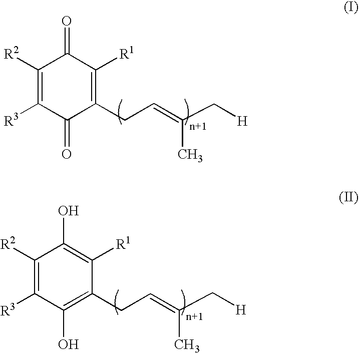

In Formula (I) and Formula (II), the integer n is selected from 0 to 13. R¹, R² and R³ are members independently selected from H, substituted or unsubstituted alkyl and substituted or unsubstituted alkoxy. R² and R³, together with the carbon atoms to which they are attached, are optionally joined to form a 5- to 7-membered ring. In a preferred embodiment, n is 9. In another preferred embodiment, R¹ is methyl, and R² and R³ are methoxy. In a particularly preferred embodiment, the ubiquinone of the invention is CoQ₁₀. A particularly preferred ubiquinol is ubiquinol-50, or reduced CoQ₁₀. Also within the scope of the current invention are formulations including both an ubiquinone and an ubiquinol.

In an exemplary embodiment, the first formulation of the invention is encompassed within a soft gelatin (soft gel) capsule. Typically, the formulations include from about 10% to about 30% (w/w) bioactive lipophilic molecule, from about 15% to about 40% (w/w) surfactant (e.g., PTS), from about 30% to about 60% (w/w) lipophilic carrier (e.g., fish oil). In an exemplary embodiment, this formulation further comprises from about 1% to about 10% (w/w) viscosity enhancer (e.g., beeswax). In an exemplary embodiment, the soft gel capsule of the invention includes a bioactive lipophilic molecule, PTS, and a lipophilic carrier (e.g., fish oil) enriched with omega fatty acid. In an exemplary embodiment, the soft gel capsule of the invention includes a bioactive lipophilic molecule, PTS, beeswax and a lipophilic carrier (e.g., fish oil) enriched with omega fatty acid.

The compositions of the invention contain from about 5% to about 50% by weight of ubiquinone/ubiquinol. In an exemplary embodiment, the composition contains from about 10% to about 30% (w/w) ubiquinone/ubiquinol, preferably from about 15% to about 25% (w/w). The soft gel capsules of the invention include ubiquinone/ubiquiniol from about 1% to about 30% by weight. In one embodiment the soft gel capsule includes from about 3% to about 20% (w/w), and preferably from about 5% to about 20% of active component, such as CoQ₁₀.

Ubiquinones/ubiquinols can be purchased commercially from sources such as Kaneka (Japan) and Nisshin (Japan). Ubiquinone/ubiquinols can also be synthesized through methods described in U.S. Pat. Nos. 6,545,184, 6,852,895 and U.S. patent application Ser. Nos. 10/992,270, 11/003,544, 11/304,023 and 10/581,566 and U.S. Provisional Patent Application No. 60/804,920.

In an exemplary embodiment, the bioactive lipophilic molecule is asiatic acid. In another exemplary embodiment, said asiatic acid is present in an amount of from about 100 mg to about 500 mg. In an exemplary embodiment, the bioactive lipophilic molecule is ursolic acid. In another exemplary embodiment, said ursolic acid is present in an amount of from about 100 mg to about 500 mg. In an exemplary embodiment, the bioactive lipophilic molecule is lutein. In another exemplary embodiment, said lutein is present in an amount of from about 5 mg to about 50 mg. In an exemplary embodiment, the bioactive lipophilic molecule is an astaxanthin. In another exemplary embodiment, said astaxanthin is present in an amount of from about 2 mg to about 20 mg. In an exemplary embodiment, the bioactive lipophilic molecule is curcumin. In another exemplary embodiment, said curcumin is present in an amount of about 500 mg. In an exemplary embodiment, the bioactive lipophilic molecule is a beta-carotene. In another exemplary embodiment, said beta-carotene is present in an amount of about 25,000 IU. In an exemplary embodiment, the bioactive lipophilic molecule is lycopene. In another exemplary embodiment, said lycopene is present in an amount of from about 5 mg to about 50 mg. In an exemplary embodiment, the bioactive lipophilic molecule is resveratrol. In another exemplary embodiment, said resveratrol is present in an amount of from about 10 mg to about 250 mg. In an exemplary embodiment, the bioactive lipophilic molecule is lecithin. In another exemplary embodiment, said lecithin is present in an amount of about 5 mg. In an exemplary embodiment, the bioactive lipophilic molecule is L-carnitine (or acetyl derivative). In another exemplary embodiment, said L-carnitine (or acetyl derivative) is present in an amount of about 500 mg. In an exemplary embodiment, the bioactive lipophilic molecule is a tocotrienol. In another exemplary embodiment, said tocotrienol is present in an amount of from about 10 mg to about 200 mg. In an exemplary embodiment, the bioactive lipophilic molecule is an alpha-lipoic acid. In another exemplary embodiment, said alpha-lipoic acid is present in an amount of from about 50 mg to about 800 mg. In an exemplary embodiment, the bioactive lipophilic molecule is salmon oil. In another exemplary embodiment, said salmon oil is present in an amount of from about 100 mg to about 2000 mg. In an exemplary embodiment, the bioactive lipophilic molecule is grape seed extract. In another exemplary embodiment, said grape seed extract is present in an amount of from about 20 mg to about 300 mg. In an exemplary embodiment, the bioactive lipophilic molecule is a bilberry extract. In another exemplary embodiment, said bilberry extract is present in an amount of from about 10 mg to about 500 mg. In an exemplary embodiment, the bioactive lipophilic molecule is flaxseed oil. In another exemplary embodiment, said flaxseed oil is present in an amount of from about 100 mg to about 2000 mg. In an exemplary embodiment, the bioactive lipophilic molecule is garlic oil. In another exemplary embodiment, said garlic oil is present in an amount of about 5 mg. In an exemplary embodiment, the bioactive lipophilic molecule is ginkgo biloba extract. In another exemplary embodiment, said ginkgo biloba extract is present in an amount of from about 50 mg to about 500 mg. In an exemplary embodiment, the bioactive lipophilic molecule is pumpkin seed oil. In another exemplary embodiment, said pumpkin seed oil is present in an amount of from about 100 mg to about 2000 mg. In an exemplary embodiment, the bioactive lipophilic molecule is green tea catechins extract. In another exemplary embodiment, said green tea catechins extract is present in an amount of from about 50 mg to about 500 mg. In an exemplary embodiment, the bioactive lipophilic molecule is kava. In another exemplary embodiment, said kava is present in an amount of about 5 mg. In an exemplary embodiment, the bioactive lipophilic molecule is evening primrose oil. In another exemplary embodiment, said evening primrose oil is present in an amount of from about 100 mg to about 2000 mg. In an exemplary embodiment, the bioactive lipophilic molecule is wheat germ oil. In another exemplary embodiment, said wheat germ oil is present in an amount of from about 100 mg to about 1000 mg. In an exemplary embodiment, the bioactive lipophilic molecule is hyaluronic acid. In another exemplary embodiment, said hyaluronic acid is present in an amount of from about 10 mg to about 500 mg. In an exemplary embodiment, the bioactive lipophilic molecule is saw palmetto berry oil extract. In another exemplary embodiment, said saw palmetto berry oil extract is present in an amount of from about 50 mg to about 500 mg. In an exemplary embodiment, the bioactive lipophilic molecule is ginseng. In another exemplary embodiment, said ginseng is present in an amount of from about 100 mg to about 1000 mg. In an exemplary embodiment, the bioactive lipophilic molecule is Japanese knotwood extract. In another exemplary embodiment, said Japanese knotwood extract is present in an amount of from about 10 mg to about 500 mg. In an exemplary embodiment, the bioactive lipophilic molecule is phytosterol. In another exemplary embodiment, said phytosterol is present in an amount of from about 50 mg to about 1000 mg. In an exemplary embodiment, the bioactive lipophilic molecule is hawthorne. In another exemplary embodiment, said hawthorne is present in an amount of from about 50 mg to about 1000 mg. In an exemplary embodiment, the bioactive lipophilic molecule is St. John's wort. In another exemplary embodiment, said St. John's wort is present in an amount of from about 50 mg to about 1000 mg. In an exemplary embodiment, the bioactive lipophilic molecule is melatonin. In another exemplary embodiment, said melatonin is present in an amount of from about 50 mg to about 1000 mg. In an exemplary embodiment, the bioactive lipophilic molecule is valerian. In another exemplary embodiment, said valerian is present in an amount of from about 50 mg to about 1000 mg. In an exemplary embodiment, the bioactive lipophilic molecule is yohimbe. In another exemplary embodiment, said yohimbe is present in an amount of from about 50 mg to about 1000 mg. In an exemplary embodiment, the bioactive lipophilic molecule is ephedra. In another exemplary embodiment, said ephedra is present in an amount of from about 50 mg to about 1000 mg. In an exemplary embodiment, the bioactive lipophilic molecule is red clover. In another exemplary embodiment, said red clover is present in an amount of from about 100 mg to about 1000 mg. In an exemplary embodiment, the bioactive lipophilic molecule is cayenne. In another exemplary embodiment, said cayenne is present in an amount of from about 100 mg to about 1000 mg. In an exemplary embodiment, the bioactive lipophilic molecule is echinacea. In another exemplary embodiment, said echinacea is present in an amount of from about 100 mg to about 1000 mg. In an exemplary embodiment, the bioactive lipophilic molecule is arnica Montana. In another exemplary embodiment, said arnica Montana is present in an amount of from about 100 mg to about 1000 mg. In an exemplary embodiment, the bioactive lipophilic molecule is docosahexaenoic acid. In another exemplary embodiment, wherein said docosahexaenoic acid is present in an amount of from about 50 mg to about 1000 mg. In an exemplary embodiment, the bioactive lipophilic molecule is eicosapentaenoic acid (EPA). In another exemplary embodiment, wherein said eicosapentaenoic acid is present in an amount of from about 50 mg to about 1000 mg. In an exemplary embodiment, the bioactive lipophilic molecule is α-linolenic acid (ALA). In another exemplary embodiment, wherein said α-linolenic acid is present in an amount of from about 50 mg to about 1000 mg.

In another exemplary embodiment, the formulation is mixed in water, rather then being encompassed within a soft gelatin (soft gel) capsule. This formulation is a member selected from: (a) a bioactive lipophilic molecule which is a member selected from lecithin, garlic oil, kava, and combinations thereof, (b) a surfactant described herein; and a (c) a lipophilic carrier described herein. In an exemplary embodiment, this formulation further comprises (d) a viscosity enhancer described herein. In another exemplary embodiment, about 5 g of the bioactive lipophilic molecule is present in the aqueous formulation.

### III. (b) Surfactant

The compositions of the invention can contain one or more surfactant. The term surfactant as used herein includes any natural or unnatural emulsifier. In an exemplary embodiment, the surfactant has a structure according to Formula (III):

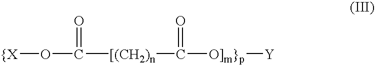

wherein X is a residue of a hydrophobic moiety, Y is a residue of a hydrophilic moiety, p is 1 or 2, m is 0 or 1, and n is an integer greater than or equal to 0. The hydrophobic moiety of the surfactant is a hydrophobic (lipophilic) molecule having an esterifiable hydroxy group and is preferably a sterol or a tocopherol or a ubiquinone/ubiquinol. In an exemplary embodiment, the hydrophobic molecule is a member selected from CoQ₁₀, cholesterol, 7-dehydrocholesterol, campesterol, sitosterol, ergosterol, stigmasterol, or a tocopherol (alpha, beta, delta, and gamma) and four compounds of tocotrienols (alpha, beta, delta, and gamma. Cholesterol and sitosterol are preferred sterols, sitosterol being particularly preferred. a-(+)-Tocopherol and a-(+−)-tocopherol are preferred tocopherols, a-(+)-tocopherol (vitamin E) being particularly preferred. The residue of the hydrophobic moiety is the entire hydrophobic molecule, except for its esterified hydroxy group, such as 3-b-hydroxy group of cholesterol or sitosterol or 6-hydroxy group of a-tocopherol.

The hydrophilic moiety of the surfactant is a hydrophilic molecule having an esterifiable hydroxy or carboxy group, and is preferably a member selected from polyalcohols, polyethers, polyanions, polycations, polyphosphoric acids, polyamines, polysaccharides, polyhydroxy compounds, polylysines, and derivatives thereof. Of those, polyethers are preferred, polyalkylene glycols being particularly preferred. The term “polyalkylene glycol” includes polymers of lower alkylene oxides, in particular polymers of ethylene oxide (polyethylene glycols) and propylene oxide (polypropylene glycols), having an esterifiable hydroxy group at least at one end of the polymer molecule, as well as derivatives of such polymers having esterifiable carboxy groups. The residue of the hydrophilic moiety is the entire hydrophilic molecule, except for its esterified hydroxy or carboxy group or groups, such as terminal hydroxy groups of a polyethylene glycol molecule.

Polyethylene glycols are most particularly preferred for the practice of the present invention. Suitable polyethylene glycols may have a free hydroxy group at each end of the polymer molecule, or may have one hydroxy group etherified with a lower alkyl, e.g., a methyl group. Also suitable for the practice of the invention are derivatives of polyethylene glycols having esterifiable carboxy groups. Polyethylene glycols are commercially available under the trade name PEG, usually as mixtures of polymers characterized by an average molecular weight. Polyethylene glycols having an average molecular weight from about 300 to about 5000 are preferred, those having an average molecular weight from about 600 to about 1000 being particularly preferred.

Compounds of formula (III) for which m is equal to 1 can be regarded as diesters of an alkanedioic acid of the general formula HOOC—(CH₂)ₙ—COOH. For the practice of the present invention, alkanedioic acids with n from 0 to 18 are preferred, those with n from 6 to 10 being particularly preferred. Sebacic acid (n=8) is most particularly preferred.

In a preferred embodiment, the surfactant is a member selected from polyoxyethanyl-tocopherol-sebacate (PTS), polyoxyethanyl-sitosterol-sebacate (PSS), polyoxyethanyl-cholesterol-sebacate (PCS), polyoxyethanyl-ubiquinol-sebacate (PQS) and combinations thereof.

In an exemplary embodiment, the formulations of the invention include from about 10% to about 50% by weight of a surfactant, such as PTS. Preferably, the formulations include from about 15% to about 40% (w/w) surfactant, more preferably from about 20% to about 40% (w/w), and even more preferably from about 20 to about 35% (w/w). The soft gel capsules of the invention (based on a soft gel capsule weight of from about 900 mg to about 1200 mg) include a surfactant from about 1% to about 30% by weight. In one embodiment, the soft gel capsule includes from about 5% to about 30% (w/w), preferably from about 8% to about 20% of a surfactant, such as PTS.

In another exemplary embodiment, the surfactant is added to the compositions of the invention at a ratio of ubiquinone/ubiquinol to surfactant from about 1:1 to about 1:5 (w/w), preferably from about 1:1 to about 1:3 (w/w), and more preferably from about 1:1 to about 1:2 (w/w). In another exemplary embodiment, in which CoQ₁₀ is formulated in a composition including PTS as the surfactant, and rice bran oil, fish oil or omega fatty acid enriched versions thereof as the lipophilic carrier, the ratio of CoQ₁₀ to PTS is preferably from about 1:1 to about 1:2 (w/w) and, more preferably, about 1:1.5 (w/w).

Surfactants utilized in the compositions of the invention include those described in U.S. Provisional Patent Application 60/773,951; and U.S. Pat. Nos. 6,045,826; 6,191,172 and 6,632,443 to Borowy-Borowski et al., which are incorporated herein by reference for all purposes. These surfactants can be purchased commercially from sources such as Zymes (New Jersey) or produced according to the methods described in the above documents.

### Addition of Surfactant Reduces Formulation Particle Size in Aqueous Solution

In an exemplary embodiment, the surfactant contributes to the formation of micelles once the composition is added to an aqueous solution. The size of the formed micelles in solution (particle size) may be measured using a dynamic light scattering (DLS) detector (compare Example 2). Typically, smaller particle sizes are associated with a greater tendency of the human body to absorb the active ingredient contained in those particles or micelles. Thus, in one embodiment, the ubiquinone/ubiquinol compositions of the invention form particle sizes in aqueous solution, which are decreased when compared with the particle sizes formed by known formulations. The average particle size formed by the compositions of the invention in aqueous solution is preferably below about 100 nm. In exemplary embodiment, the average particle size is from about 10 nm to about 90 nm. An exemplary average particle size is from about 5 nm to about 70 nm, preferably from about 10 nm to about 50 nm, more preferably from about 10 nm to about 30 nm, and most preferably from about 15 nm to about 25 nm.

### III. (c) Lipophilic Carrier

The compositions of the invention can contain any lipophilic compound, such as, for example, monoglycerides, diglycerides, triglycerides, phosphatides, cerebrosides, sterols, terpenes, fatty alcohols, fatty acids and combinations thereof. Suitable lipophilic carriers of the invention include, but are not limited to, for example, natural or synthetic oils, fats, waxes or combinations thereof. Moreover, the fatty acids can be derived, without limitation, from non-hydrogenated oils, partially hydrogenated oils, fully hydrogenated oils or combinations thereof. Non-limiting exemplary sources of fatty acids (their esters and salts) include seed oil, food grade oil, fish or marine oil, canola oil, vegetable oil, safflower oil, sunflower oil, nasturtium seed oil, mustard seed oil, olive oil, sesame oil, soybean oil, corn oil, peanut oil, cottonseed oil, rice bran oil, babassu nut oil, palm oil, low erucic rapeseed oil, palm kernel oil, lupin oil, coconut oil, flaxseed oil, evening primrose oil, jojoba, tallow, beef tallow, butter, chicken fat, lard, dairy butterfat, shea butter or combinations thereof.

In an exemplary embodiment, the lipophilic carrier is a member selected from an essential oil, an essential oil component, fish oil, vegetable oil, rice bran oil, soybean oil, an oil comprising an omega-3 fatty acid, an oil comprising an omega-6 fatty acid, an oil comprising an omega-9 fatty acid, an oil comprising an omega-12 fatty acid and combinations thereof. In an exemplary embodiment, the lipophilic carrier comprises an essential fatty acid (EFA). In another exemplary embodiment, the EFA is a member selected from linoleic acid, alpha-linolenic acid, docosahexaenoic acid, arachidonic acid and oleic acid. For examples of omega-3, omega-6, omega-9 and omega-12 fatty acids, which are useful in the formulations of the invention, compare Tables 1-3, below.

In an exemplary embodiment, the omega fatty acid is part of the lipophilic carrier of the invention, such as fish oil and flax seed oil. In another exemplary embodiment, the lipophilic carrier is enriched with omega fatty acid. In yet another exemplary embodiment, the lipophilic carrier is an oil, which is enriched with omega-3 fatty acid, such as alpha-linolenic acid. In an exemplary embodiment, the lipophilic carrier is an oil comprising an omega-3 fatty acid. In an exemplary embodiment, the lipophilic carrier is an oil comprising an omega-3 fatty acid which is included in Table 1. In an exemplary embodiment, the omega-3 fatty acid is alpha-linolenic acid. In an exemplary embodiment, the lipophilic carrier is an oil comprising an omega-6 fatty acid. In an exemplary embodiment, the lipophilic carrier is an oil comprising an omega-6 fatty acid which is included in Table 2. In an exemplary embodiment, the omega-6 fatty acid is a member selected from linoleic acid and arachidonic acid. In an exemplary embodiment, the lipophilic carrier is an oil comprising an omega-9 fatty acid. In an exemplary embodiment, the lipophilic carrier is an oil comprising an omega-9 fatty acid which is included in Table 3. In an exemplary embodiment, the omega-9 fatty acid is oleic acid. In an exemplary embodiment, the lipophilic carrier is an oil comprising an omega-12 fatty acid.

TABLE 1
Important Omega-3 Fatty Acids in Human Nutrition

| Common Name | Lipid Name | Chemical Name |
|---|---|---|
| α-Linolenic acid | 18:3 (n-3) | octadeca-9,12,15-trienoic acid |
| (ALA) | | |
| Stearidonic acid | 18:4 (n-3) | octadeca-6,9,12,15-tetraenoic |
| | | acid |
| Eicosatetraenoic | 20:4 (n-3) | eicosa-8,11,14,17-tetraenoic |
| acid | | acid |
| Eicosapentaenoic | 20:5 (n-3) | eicosa-5,8,11,14,17-pentaenoic |
| acid (EPA) | | acid |
| Docosapentaenoic | 22:5 (n-3) | docosa-7,10,13,16,19-pentaenoic |
| acid | | acid |
| Docosahexaenoic | 22:6 (n-3) | docosa-4,7,10,13,16,19-hexaenoic |
| acid (DHA) | | acid |

TABLE 2
Important Omega-6 Fatty Acids in Human Nutrition

| Common Name | Lipid Name | Chemical Name |
|---|---|---|
| Linoleic acid | 18:2 (n-6) | 9,12-octadecadienoic acid |
| Gamma-linolenic acid | 18:3 (n-6) | 6,9,12-octadecatrienoic acid |
| Eicosadienoic acid | 20:2 (n-6) | 11,14-eicosadienoic acid |
| Dihomo-gamma-linolenic | 20:3 (n-6) | 8,11,14-eicosatrienoic acid |
| acid | | |
| Arachidonic acid | 20:4 (n-6) | 5,8,11,14-eicosatetraenoic acid |
| Docosadienoic acid | 22:2 (n-6) | 13,16-docosadienoic acid |
| Adrenic acid | 22:4 (n-6) | 7,10,13,16-docosatetraenoic |
| | | acid |
| Docosapentaenoic acid | 22:5 (n-6) | 4,7,10,13,16-docosapentaenoic |
| | | acid |

TABLE 3
Important Omega-9 Fatty Acids in Human Nutrition

| Common Name | Lipid Name | Chemical Name |
|---|---|---|
| oleic acid | 18:1 (n-9) | 9-octadecenoic acid |
| eicosenoic acid | 20:1 (n-9) | 11-eicosenoic acid |
| mead acid | 20:3 (n-9) | 5,8,11-eicosatrienoic acid |
| erucic acid | 22:1 (n-9) | 13-docosenoic acid |
| nervonic acid | 24:1 (n-9) | 15-tetracosenoic acid |

Excessive amounts of omega-6 polyunsaturated fatty acids and a very high omega-6/omega-3 ratio has been linked with pathogenesis of many diseases, including cardiovascular disease, cancer, and inflammatory and autoimmune diseases. The ratio of omega-6 to omega-3 in modern diets is approximately 15:1, whereas ratios of 2:1 to 4:1 have been associated with reduced mortality from cardiovascular disease, suppressed inflammation in patients with rheumatoid arthritis, and decreased risk of breast cancer.

Thus, in one embodiment, the formulations of the invention include a health-beneficial ratio of omega-3 to omega-6 fatty acids. Preferably, the ratio between omega-6 fatty acid and omega-3 fatty acid is from about 2:1 to about 10:1, more preferably from about 3:1 to about 5:1, and even more preferably from about 4:1 to about 2:1.

In an exemplary embodiment, the lipophilic carrier is a fish or marine oil. In another exemplary embodiment, fish or marine oil sources include shellfish oil, tuna oil, mackerel oil, salmon oil, menhaden, anchovy, herring, trout, sardines or combinations thereof. In another exemplary embodiment, the fish or marine oil is enriched with omega-3 or omega-6 fatty acids.

In an exemplary embodiment, the lipophilic carrier comprises a member selected from an essential oil and an essential oil component. In an exemplary embodiment, the essential oil component is a monoterpene. CoQ₁₀ formulations including monoterpenes and limonene in particular are described, for instance, in US Patent Application 2005/0287206, which is incorporated herein by reference in its entirety. Suitable examples of monoterpenes include, but are not limited to, limonene, pinene, cintronellol, terpinene, nerol, menthane, carveol, S-linalool, safrol, cinnamic acid, apiol, geraniol, thymol, citral, carvone, camphor, and the like and derivatives thereof. For information about the structure and synthesis of terpenes, including terpenes of the invention, see Kirk-Othmer Encyclopedia of Chemical Technology, Mark, et al., eds., 22:709-762 3d Ed (1983), the teachings of which are incorporated herein in their entirety.

A particularly preferred monoterpene of the invention is a member selected from R-(+)-limonene, S-(−)-limonene and mixtures thereof. Suitable limonene derivatives include perillyl alcohol, perillic acid, cis-dihydroperillic acid, trans-dihydroperillic acid, methyl esters of perillic acid, methyl esters of dihydroperillic acid, limonene-2-diol, uroterpenol, and combinations thereof.

The formulations of the invention can include from about 10% to about 80% by weight of a lipophilic carrier described herein. In an exemplary embodiment, the formulations of the invention include from about 20% to about 70% (w/w) lipophilic carrier, preferably from about 30% to about 60% (w/w), and, more preferably, from about 33% to about 50% (w/w). The soft gel capsules of the invention (based on a soft gel capsule weight of from about 900 mg to about 1200 mg) include lipophilic carrier from about 5% to about 60% by weight. In one embodiment, the soft gel capsule includes from about 5% to about 40% (w/w), preferably from about 10% to about 35% of a lipophilic carrier, such as fish oil.

In an exemplary embodiment, the lipophilic carrier is a member selected from medium chain triglycerides and soy lechitin.

The lipophilic carriers of the invention can be purchased commercially from sources such as BI International and Soft Gel Technologies.

The Addition of Omega Fatty Acid Reduces the Necessary Amount of Viscosity Enhancer and/or Surfactant

In one embodiment, the omega fatty acid (e.g., omega-3 fatty acid) can function as a viscosity enhancer within the formulation of the invention. Hence, in an exemplary embodiment, the addition of omega fatty acid or an omega fatty acid enriched oil to the composition of the invention, eliminates the need for or reduces the amount of viscosity enhancer (e.g., bees wax) and/or the amount of surfactant (e.g., PTS) needed to produce a softgel capsule with sufficient stability.

### III. (d) Viscosity Enhancer

The compositions and soft gelatin capsules of the invention can contain one or more compounds, which are useful for the modification of viscosity. In an exemplary embodiment, in which the composition of the invention is part of a soft gel capsule, a particular viscosity of the composition may be necessary, for instance, to prevent the components from separating within the capsule and obtain stability. In a preferred embodiment, the viscosity modifier/enhancer is a member selected from beeswax and other waxes. Beeswax in this invention can be either filtered (to remove unwanted/immunogenic particles) or unfiltered. In a preferred embodiment, the beeswax used in the formulations described herein is filtered.

The formulations of the invention can include from about 5% to about 30% by weight of a viscosity enhancer described herein. In an exemplary embodiment, the formulations of the invention include from about 1% to about 15% (w/w) of a viscosity enhancer, preferably from about 1% to about 10% (w/w). The soft gel capsules of the invention (based on a soft gel capsule weight of from about 900 mg to about 1200 mg) include a viscosity enhancer from about 0.1% to about 10% by weight. In one embodiment, the soft gel capsule includes from about 1% to about 10% (w/w), preferably from about 1% to about 8% of a viscosity enhancer, such as beeswax.

### III. (e) Other Components

The compositions and soft gel formulations of the invention can further include various ingredients to help stabilize the composition, or help promote the bioavailability of the ubiquinone/ubiquinol, or serve as additional nutrients to an individual's diet. Suitable additives can include vitamins and biologically-acceptable minerals. Non-limiting examples of vitamins include vitamin A, B vitamins, vitamin C, vitamin D, vitamin E, vitamin K and folic acid. Non-limiting examples of minerals include iron, calcium, magnesium, potassium, copper, chromium, zinc, molybdenum, iodine, boron, selenium, manganese, derivatives thereof or combinations thereof. These vitamins and minerals may be from any source or combination of sources, without limitation. Non-limiting exemplary B vitamins include, without limitation, thiamine, niacinamide, pyridoxine, riboflavin, cyanocobalamin, biotin, pantothenic acid or combinations thereof.

Various additives can be incorporated into the present compositions. Optional additives of the present formulations include, without limitation, pharmaceutical drug molecules, antibiotics, sterols, vitamins, provitamins, carotenoids (e.g., alpha and beta-carotenes, cryptoxanthin, lutein and zeaxanthin), phospholipids, L-carnitine, starches, sugars, fats, antioxidants, reducing agents, free radical scavengers, amino acids, amino acid analogs, proteins, solvents, emulsifiers, adjuvants, sweeteners, fillers, flavoring agents, coloring agents, lubricants, binders, moisturizing agents, preservatives, suspending agents, starch, hydrolyzed starch(es), derivatives thereof and combinations thereof.

In an exemplary embodiment, the formulation further comprises gelatin. In an exemplary embodiment, the formulation further comprises sorbitol. In an exemplary embodiment, the formulation further comprises glycerin. In an exemplary embodiment, the formulation further comprises purified water. In an exemplary embodiment, the formulation further comprises polysorbate 80. In an exemplary embodiment, the formulation further comprises hydroxylated lechitin. In an exemplary embodiment, the formulation further comprises medium chain triglycerides. In an exemplary embodiment, the formulation further comprises annato seed extract. In an exemplary embodiment, the formulation further comprises soybean oil. In an exemplary embodiment, the formulation further comprises omega-3 enriched fish oil. In an exemplary embodiment, the formulation further comprises rice bran oil. In an exemplary embodiment, the formulation further comprises carotenoids. In an exemplary embodiment, the formulation further comprises titanium dioxide. In an exemplary embodiment, the formulation further comprises suspending agents such as silica (silicon dioxide). In an exemplary embodiment, the formulation further comprises riboflavin. Various other additives can be incorporated into the present formulations including, without limitation, phospholipids, L-carnitine, anti-inflammatory agents, anti-aging agents, starches, sugars, fats, antioxidants, amino acids, proteins, flavorings, coloring agents, hydrolyzed starch(es) and derivatives thereof (such as time release esters (Ester-C, Ester-E)) or combinations thereof. Anti-inflammatory agents of use in the invention include, but are not limited to, bisabolol, mentholatum, dapsone, aloe, hydrocortisone, and the like. Anti-aging agents of use in the invention include, but are not limited to, niacinamide, retinol and retinoid derivatives, AHA, ascorbic acid, lipoic acid, beta hydroxy acids, salicylic acid, copper binding peptides and the like.

The formulations described herein can also include vitamins and biologically-acceptable minerals. Non-limiting examples of vitamins include vitamin A, B vitamins, vitamin C, vitamin D, vitamin E, vitamin K and folic acid. Vitamin derivatives can also be added to the formulations of the invention, such as tazarotene, calcipotriene, tretinoin, adapalene and the like. The Vitamin E family includes a family of four compounds (forms) of tocopherols (alpha, beta, delta, and gamma) and four compounds of tocotrienols (alpha, beta, delta, and gamma). Non-limiting examples of minerals include iron, calcium, magnesium, potassium, copper, chromium, zinc, molybdenum, iodine, boron, selenium, manganese, derivatives thereof or combinations thereof. These vitamins and minerals may be from any source or combination of sources, without limitation. Non-limiting exemplary B vitamins include, without limitation, thiamine, niacinamide, pyridoxine, riboflavin, cyanocobalamin, biotin, pantothenic acid or combinations thereof.

Vitamin(s), if present, are present in the composition of the invention in an amount ranging from about 5 mg to about 500 mg. More particularly, the vitamin(s) is present in an amount ranging from about 10 mg to about 400 mg. Even more specifically, the vitamin(s) is present from about 250 mg to about 400 mg. Most specifically, the vitamin(s) is present in an amount ranging from about 10 mg to about 50 mg. For example, B vitamins are usually incorporated in the range of about 1 milligram to about 10 milligrams, i.e., from about 3 micrograms to about 50 micrograms of B12. Folic acid, for example, is generally incorporated in a range of about 50 to about 400 micrograms, biotin is generally incorporated in a range of about 25 to about 700 micrograms and cyanocobalamin is incorporated in a range of about 3 micrograms to about 50 micrograms.

Mineral(s), if present, are present in the composition of the invention in an amount ranging from about 25 mg to about 1000 mg. More particularly, the mineral(s) are present in the composition ranging from about 25 mg to about 500 mg. Even more particularly, the mineral(s) are present in the composition in an amount ranging from about 100 mg to about 600 mg.

In the formulations of the invention the additional components are usually a minor component (from about 0.001% to about 20% by weight or preferably from about 0.01% to about 10% by weight) with the remainder being various vehicles or carriers and processing aids helpful for forming the desired dosing form.

### IV. Second Formulations

The formulations of the invention can take a variety of forms adapted to the chosen route of administration. Those skilled in the art will recognize various synthetic methodologies that may be employed to prepare non-toxic pharmaceutical formulations incorporating the compounds described herein. Those skilled in the art will recognize a wide variety of non-toxic pharmaceutically acceptable solvents that may be used to prepare solvates of the compounds of the invention, such as water, ethanol, propylene glycol, mineral oil, vegetable oil and dimethylsulfoxide (DMSO).

The compositions of the invention may be administered orally, topically, parenterally, by inhalation or spray or rectally in dosage unit formulations containing conventional non-toxic pharmaceutically acceptable carriers, adjuvants and vehicles. It is further understood that the best method of administration may be a combination of methods. Oral administration in the form of a pill, capsule, soft gel capsule, elixir, syrup, lozenge, troche, or the like is particularly preferred. The term parenteral as used herein includes subcutaneous injections, intradermal, intravascular (e.g., intravenous), intramuscular, spinal, intrathecal injection or like injection or infusion techniques.

The formulations are preferably in a form suitable for oral use, for example, as tablets, troches, lozenges, aqueous or oily suspensions, dispersible powders or granules, emulsion, hard or soft capsules, soft gel capsules, or syrups or elixirs.

The formulations described herein may be prepared according to any method known in the art for the manufacture of pharmaceutical formulations and nutriceuticals, and such compositions may contain one or more agents selected from the group consisting of sweetening agents, flavoring agents, coloring agents and preserving agents in order to provide pharmaceutically elegant and palatable preparations. Tablets may contain the active ingredient in admixture with non-toxic pharmaceutically acceptable excipients that are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia; and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Aqueous suspensions contain the active materials in admixture with excipients suitable for the manufacture of aqueous suspensions. Such excipients are suspending agents, for example sodium carboxymethylcellulose, methylcellulose, hydroxypropylmethylcellulose, sodium alginate, polyvinylpyrrolidone, gum tragacanth and gum acacia; and dispersing or wetting agents, which may be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions may be formulated by suspending the active ingredients in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide palatable oral preparations. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

Formulations of the invention may also be in the form of oil-in-water emulsions and water-in-oil emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth; naturally-occurring phosphatides, for example soy bean, lecithin, and esters or partial esters derived from fatty acids and hexitol; anhydrides, for example sorbitan monooleate; and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative, and flavoring and coloring agents. The formulations may be in the form of a patch, sterile injectable aqueous or oleaginous suspension. This suspension may be formulated according to the known art using those suitable dispersing or wetting agents and suspending agents, which have been mentioned above. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

For administration to non-human animals, the formulations of the invention may be added to the animal's feed or drinking water. Also, it will be convenient to formulate animal feed and drinking water products so that the animal takes in an appropriate quantity of the compound in its diet. It will further be convenient to present the compound in a composition as a premix for addition to the feed or drinking water. The composition can also be added as a food or drink supplement for humans.

Dosage levels of the order of from about 5 mg to about 250 mg per kilogram of body weight per day and more preferably from about 25 mg to about 150 mg per kilogram of body weight per day, are useful in the treatment of the above-indicated conditions. The amount of active ingredient that may be combined with the carrier materials to produce a single dosage form will vary depending upon the condition being treated and the particular mode of administration. Dosage unit forms will generally contain between from about 1 mg to about 500 mg of an active ingredient.

Frequency of dosage may also vary depending on the compound used and the particular disease treated. However, for treatment of most disorders, a dosage regimen of 4 times daily or less is preferred. It will be understood, however, that the specific dose level for any particular patient will depend upon a variety of factors including the activity of the specific compound employed, the age, body weight, general health, sex, diet, time of administration, route of administration and rate of excretion, drug combination and the severity of the particular disease undergoing therapy.

The present invention also provides packaged formulations of the invention and instructions for use of the tablet, capsule, soft gel capsule, elixir, etc. Typically, the packaged formulation, in whatever form, is administered to an individual in need thereof that requires an increase in the amount of ubiquinone/ubiquinol in the individual's diet. Typically, the dosage requirement is between about 1 to about 4 dosages a day.

In an exemplary embodiment, the second formulation described herein does not contain water. In another exemplary embodiment, the second formulation described herein is essentially free of water. In another exemplary embodiment, the second formulation described herein comprises less than 1 weight percent of water. In another exemplary embodiment, the second formulation described herein comprises less than about XX weight percent of water, wherein XX is a member selected from 0.9, 0.85, 0.8, 0.75, 0.7, 0.65, 0.6, 0.55, 0.5, 0.45, 0.4, 0.35, 0.3, 0.25, 0.2, 0.15, 0.1, 0.05, 0.04, 0.03, 0.02, 0.01.

In another exemplary embodiment, the invention provides a second formulation comprising a first formulation described herein which is encapsulated by a member selected from a tablet, troche, lozenge, capsule (hard, soft or soft gel), syrup or elixir. In an exemplary embodiment, the ratio of the bioactive lipophilic molecule to the “shell” (the non-first formulation part of the second formulation) is from about 1:1 to about 1:10 (w/w), preferably from about 1:1 to about 1:6 (w/w), and more preferably from about 1:1 to about 1:4 (w/w) and more preferably from about 1:1 to about 1:3 (w/w), and more preferably about 1:3 (w/w).

### IV. a) Soft Gel Capsules

In an exemplary embodiment, the first formulation of the invention is encapsulated within a soft gel capsule. In another exemplary embodiment, the capsule is essentially free of precipitation within said capsule. In another exemplary embodiment, less than XXX weight percent of the first formulation within the second formulation is present as a precipitate, wherein XXX is a member selected from 5, 4.5, 4, 3.5, 3, 2.5, 2, 1.5, 1, and 0.5. In another exemplary embodiment, less than 5 weight percent of the first formulation within the second formulation is present as a precipitate. In another exemplary embodiment, the capsule is essentially free of precipitated ubiquinone/ubiquinol within said capsule. In another exemplary embodiment, the ubiquinone/ubiquinol is CoQ₁₀, the surfactant is PTS, the lipophilic carrier is fish or marine oil, and the viscosity enhancer is beeswax. In another exemplary embodiment, the surfactant is PTS, the lipophilic carrier is omega fatty acid enriched fish oil, and the viscosity enhancer is beeswax. In another exemplary embodiment, the ratio of said ubiquinone/ubiquinol to said PTS is from about 1:1 to about 1:2.

Soft gel or soft gelatin capsules can be prepared, for example, without limitation, by dispersing the bioactive lipophilic molecule and surfactant in an appropriate vehicle (e.g. fish or marine oil, rice bran oil, monoterpene and/or beeswax) to form a high viscosity mixture. This mixture is then encapsulated with a gelatin based film using technology and machinery known to those in the soft gel industry. The industrial units so formed are then dried to constant weight. Typically, the weight of the capsule is between about 100 to about 2500 milligrams and in particular weigh between about 1500 and about 1900 milligrams, and more specifically can weigh between about 1500 and about 2000 milligrams.

For example, when preparing soft gelatin shells, the shell can include between about 20 to 70 percent gelatin, generally a plasticizer and about 5 to about 60% by weight sorbitol. The filling of the soft gelatin capsule is primarily liquid (principally limonene, in combination with fish or marine oil, rice bran oil and/or beeswax if desired) and can include, apart from the antioxidant actives, a hydrophilic matrix. Precipitate, preferably a minimal amount, can be present in some embodiments of the invention. The hydrophilic matrix, if present, is a polyethylene glycol having an average molecular weight of from about 200 to 1000. Further ingredients are optionally thickening agents. In one embodiment, the hydrophilic matrix includes polyethylene glycol having an average molecular weight of from about 200 to 1000, 5 to 15% glycerol, and 5 to 15% by weight of water. The polyethylene glycol can also be mixed with propylene glycol and/or propylene carbonate.

In another embodiment, the soft gel capsule is prepared from gelatin, glycerin, water and various additives. Typically, the percentage (by weight) of the gelatin is between about 30 and about 50 weight percent, in particular between about 35 and about weight percent and more specifically about 42 weight percent. The formulation includes between about 15 and about 25 weight percent glycerin, more particularly between about 17 and about 23 weight percent and more specifically about 20 weight percent glycerin.

The remaining portion of the capsule can be water. The amount varies from between about 25 weight percent and about 40 weight percent, more particularly between about 30 and about 35 weight percent, and more specifically about 35 weight percent. The remainder of the capsule can vary, generally, between about 2 and about 10 weight percent composed of a flavoring agent(s), sugar, coloring agent(s), etc. or combination thereof. After the capsule is processed, the water content of the final capsule is often between about 5 and about 10 weight percent, more particularly 7 and about 12 weight percent, and more specifically between about 9 and about 10 weight percent.

As for the manufacturing, it is contemplated that standard soft shell gelatin capsule manufacturing techniques can be used to prepare the soft-shell product. Examples of useful manufacturing techniques are the plate process, the rotary die process pioneered by R. P. Scherer, the process using the Norton capsule machine, and the Accogel machine and process developed by Lederle. Each of these processes are mature technologies and are all widely available to any one wishing to prepare soft gelatin capsules.

Preparation of the soft gel capsules was accomplished by methods well known in the art including, but not limited to those described throughout the specification and in U.S. Pat. Nos. 6,616,942, 6,623,734 and pending U.S. Ser. Nos. 10/035,753 and 09/825,920, the contents of which are incorporated herein by reference in their entirety.

When a soft gel capsule is prepared, the total weight is between about 250 milligrams and about 2.5 gram in weight, e.g., 400-750 milligrams. Therefore, the total weight of additives, such as vitamins and antioxidants, is between about 80 milligrams and about 2000 milligrams, alternatively, between about 100 milligrams and about 1500 milligrams, and in particular between about 120 milligrams and about 1200 milligrams. In an exemplary embodiment, the additives include the non-soft shell gel capsule materials. In an exemplary embodiment, the additives include ubiquinone/ubiquinol [or non-CoQ10], surfactant, lipophilic carrier and viscosity enhancer. In particular, the soft gel capsule typically weighs between about 1000 milligrams and 1300 milligrams, wherein the percentage fill is about 50% of the entire weight of the capsule, i.e., from about 450 to about 800 milligrams fill weight. The fill weight includes the active ingredient(s), solubilizing agents, etc.

The presence of a surfactant described herein in the first formulation enables a substantial increase in the amount of bioactive lipophilic molecule which can be present in the second formulation. The presence of this surfactant enables the production of a second formulation with amounts of bioactive lipophilic molecule comparable to currently marketed formulations, but which is smaller and/or lighter. This improvement is desirable since smaller and/or lighter pharmaceutical or nutraceutical or cosmetic formulations are easier for patients to consume, thus increasing patient compliance with therapeutic regimens.

For example, current soft gel capsule technologies on the market which contain about 100 mg of CoQ10 have a total weight of from about 1.3 g to about 1.6 g. Examples of these technologies include Tishcon's Q-Gel (total capsule wgt: approx. 1.54 g; shell=approx. 0.70 g; contents=approx. 0.84 g, 100 mg of which is COQ10) and Soft Gel Technology's CoQ-Sol. The use of the surfactants in the current invention enable the production of a 100 mg soft gel capsule with a weight of less than about 1 g.

In an exemplary embodiment, the invention provides a second formulation described herein, wherein the weight of the second formulation is not greater than 1 gram. In an exemplary embodiment, the invention provides a second formulation described herein, wherein the weight of the second formulation is between about 900 mg and 1 g. In an exemplary embodiment, the invention provides a second formulation described herein, wherein the weight of the second formulation is between about 800 mg and 900 mg. In an exemplary embodiment, the invention provides a second formulation described herein, wherein the weight of the second formulation is between about 700 mg and 800 mg. In an exemplary embodiment, the invention provides a second formulation described herein, wherein the weight of the second formulation is between about 600 mg and 700 mg. In an exemplary embodiment, the invention provides a second formulation described herein, wherein the weight of the second formulation is between about 1.2 g and 1.3 g. In an exemplary embodiment, the invention provides a second formulation described herein, wherein the weight of the second formulation is between about 1.1 g and 1.2 g. In an exemplary embodiment, the invention provides a second formulation described herein, wherein the weight of the second formulation is between about 1.0 g and 1.1 g.

In an exemplary embodiment, when the amount of CoQ10 is between about invention provides a second formulation described herein, wherein the weight of the second formulation is between about 1.0 g and 1.1 g.

In an exemplary embodiment, the invention provides a second formulation which is a soft gel capsule containing a first formulation comprising: a) CoQ₁₀; b) PTS; c) beeswax; and d) a lipophilic carrier, wherein the weight of said second formulation is a member selected from about 1.2 g to about 1.3 g, from about 1.1 g to about 1.2 g, from about 1.0 g to about 1.1 g, from about 900 mg to about 1.0 g, from about 800 mg to about 900 mg and from about 700 mg to about 800 mg, from about 600 mg to about 700 mg, from about 500 mg to about 600 mg, from about 400 mg to about 500 mg. In an exemplary embodiment, said second formulation contains from about 1 mg to about 750 mg of CoQ₁₀. In an exemplary embodiment, said second formulation contains from about 100 mg to about 500 mg of CoQ₁₀. In an exemplary embodiment, said second formulation contains from about 100 mg to about 400 mg of CoQ₁₀. In an exemplary embodiment, said second formulation contains from about 75 mg to about 150 mg of CoQ₁₀. In an exemplary embodiment, said second formulation contains from about 25 mg to about 100 mg of CoQ₁₀. In an exemplary embodiment, said second formulation contains from about 90 mg to about 110 mg of CoQ₁₀. In an exemplary embodiment, the ratio of said CoQ10 to said PTS is a member selected from about 1:1 to about 1:5. In an exemplary embodiment, the ratio of said CoQ10 to said PTS is a member selected from about 1:1 to about 1:3. In an exemplary embodiment, the ratio of said CoQ10 to said PTS is a member selected from about 3:5 to about 3:4. In an exemplary embodiment, the ratio of said CoQ10 to said PTS is a member selected from about 2:3.

In an exemplary embodiment, the invention provides a second formulation which is a soft gel capsule containing a first formulation comprising: a) CoQ₁₀; b) PTS; c) beeswax; and d) a lipophilic carrier, wherein the weight of said CoQ10 is between about 10 mg and about 20 mg, and the weight of said second formulation is a member selected from about 75 mg to about 175 mg. In an exemplary embodiment, the weight of said CoQ10 is between about 40 mg and about 60 mg, and the weight of said second formulation is a member selected from about 320 mg to about 550 mg. In an exemplary embodiment, the weight of said CoQ10 is between about 90 mg and about 110 mg, and the weight of said second formulation is a member selected from about 700 mg to about 1 g. In an exemplary embodiment, the weight of said CoQ10 is between about 140 mg and about 160 mg, and the weight of said second formulation is a member selected from about 1.1 g to about 1.3 g. In an exemplary embodiment, the weight of said CoQ10 is between about 190 mg and about 210 mg, and the weight of said second formulation is a member selected from about 1.5 g to about 1.7 g. In an exemplary embodiment, the weight of said CoQ10 is between about 190 mg and about 310 mg, and the weight of said second formulation is a member selected from about 1.5 g to about 2.5 g. In an exemplary embodiment, the weight of said CoQ10 is between about 90 mg and about 210 mg, and the weight of said second formulation is a member selected from about 700 mg to about 1.7 g.

In another exemplary embodiment, the invention provides a second formulation comprising a first formulation comprising a) bioactive lipophilic molecule; b) surfactant; c) lipophilic carrier; and d) viscosity enhancer, wherein the first formulation is encapsulated within a soft gel capsule, and wherein the ratio of the bioactive lipophilic molecule to the “shell” (the non-first formulation part of the second formulation) is from about 1:1 to about 1:4 (w/w), the ratio of the bioactive lipophilic molecule to the surfactant is from about 1:1 to about 1:3 (w/w), the ratio of the bioactive lipophilic molecule to the lipophilic carrier is from about 1:1 to about 1:4 (w/w), the ratio of the bioactive lipophilic molecule to the viscosity enhancer is from about 1:0.25 to about 1:0.5 (w/w). In an exemplary embodiment, the surfactant is a member selected from PTS, PCS, PSS and PQS. In an exemplary embodiment, the surfactant is a member selected from PTS. In an exemplary embodiment, the viscosity enhancer is beeswax. In an exemplary embodiment, the lipophilic carrier is a member selected from rice bran oil, fish oil, medium chain triglycerides, soy lechitin, and combinations thereof. In an exemplary embodiment, the bioactive lipophilic molecule is a member selected from ubiquinone, ubiquinol, docosahexaenoic acid, eicosapentaenoic acid, α-linolenic acid (ALA), lutein, lycopene, astaxanthin, resveratrol. In an exemplary embodiment, the bioactive lipophilic molecule is CoQ10.

In another exemplary embodiment, the invention provides a second formulation comprising a first formulation comprising a) CoQ₁₀; b) PTS; c) lipophilic carrier; and d) beeswax, wherein the first formulation is encapsulated within a soft gel capsule, and wherein the ratio of the bioactive lipophilic molecule to the “shell” (the non-first formulation part of the second formulation) is from about 1:1 to about 1:4 (w/w), the ratio of the bioactive lipophilic molecule to the surfactant is from about 1:1 to about 1:3 (w/w), the ratio of the bioactive lipophilic molecule to the lipophilic carrier is from about 1:1 to about 1:4 (w/w), the ratio of the bioactive lipophilic molecule to the viscosity enhancer is from about 1:0.25 to about 1:0.5 (w/w). In another exemplary embodiment, the weight of the COQ10 is between about 50 mg and about 150 mg, and the weight of the second formulation is between about 700 mg and about 1.1 g. In another exemplary embodiment, the weight of the CoQ10 is between about 90 mg and about 110 mg, and the weight of the second formulation is between about 800 mg and about 950 mg. In another exemplary embodiment, the weight of the CoQ10 is about 100 mg, and the weight of the second formulation is between about 850 mg and about 900 mg. In another exemplary embodiment, the weight of the CoQ10 is from about 10 mg to about 20 mg, and the weight of the second formulation is between about 75 mg and about 175 mg. In another exemplary embodiment, the weight of the CoQ10 is about 50 mg, and the weight of the second formulation is between about 400 mg and about 500 mg. In another exemplary embodiment, the weight of the COQ10 is about 200 mg, and the weight of the second formulation is between about 1600 mg and about 1800 mg.

In another exemplary embodiment, the invention provides a second formulation comprising a first formulation comprising a) DHA; b) PTS; c) lipophilic carrier; and d) beeswax, wherein the first formulation is encapsulated within a soft gel capsule, and wherein the ratio of the bioactive lipophilic molecule to the “shell” (the non-first formulation part of the second formulation) is from about 1:1 to about 1:4 (w/w), the ratio of the bioactive lipophilic molecule to the surfactant is from about 1:1 to about 1:3 (w/w), the ratio of the bioactive lipophilic molecule to the lipophilic carrier is from about 1:1 to about 1:4 (w/w), the ratio of the bioactive lipophilic molecule to the viscosity enhancer is from about 1:0.25 to about 1:0.5 (w/w). In another exemplary embodiment, the weight of the DHA is between about 50 mg and about 150 mg, and the weight of the second formulation is between about 700 mg and about 1.1 g.

### III. (g) Additional Formulations

In another aspect, the formulation comprises (a) a ubiquinone/ubiquinol; (b) a surfactant which is a member selected from PTS, PSS, PCS, and PQS. In an exemplary embodiment, the formulation further comprises gelatin. In an exemplary embodiment, the formulation further comprises sorbitol. In an exemplary embodiment, the formulation further comprises glycerin. In an exemplary embodiment, the formulation further comprises purified water. In an exemplary embodiment, the formulation further comprises polysorbate 80. In an exemplary embodiment, the formulation further comprises hydroxylated lechitin. In an exemplary embodiment, the formulation further comprises medium chain triglycerides. In an exemplary embodiment, the formulation further comprises annato seed extract. In an exemplary embodiment, the formulation further comprises soybean oil. In an exemplary embodiment, the formulation further comprises omega-3 enriched fish oil.

In another aspect, the formulation comprises (a) a ubiquinone/ubiquinol; (b) a surfactant which is a member selected from PTS, PSS, PCS, and PQS; gelatin; sorbitol; glycerin; purified water; polysorbate 80; hydroxylated lechitin; medium chain triglycerides, annato seed extract and soybean oil. This formulation can optionally further include a vitamin and/or preservative, such as vitamin E. In another exemplary embodiment, the formulation comprises (a) a ubiquinone/ubiquinol; (b) PTS; gelatin; sorbitol; glycerin; purified water; polysorbate 80; hydroxylated lechitin; medium chain triglycerides, annato seed extract, and omega-3 enriched fish oil.

In another aspect, the formulation comprises (a) a ubiquinone/ubiquinol; (b) a surfactant which is a member selected from PTS, PSS, PCS, and PQS. In an exemplary embodiment, the formulation further comprises titanium dioxide. In an exemplary embodiment, the formulation further comprises riboflavin.

In another aspect, the formulation comprises (a) a ubiquinone/ubiquinol; (b) a surfactant which is a member selected from PTS, PSS, PCS, and PQS; soybean oil; gelatin; glycerin; beeswax; lechitin; titanium dioxide; and riboflavin. In another exemplary embodiment, the formulation comprises (a) a ubiquinone/ubiquinol; (b) PTS; gelatin; sorbitol; glycerin; purified water; polysorbate 80; hydroxylated lechitin; medium chain triglycerides, annato seed extract, and omega-3 enriched fish oil.

In another aspect, the formulation comprises (a) a ubiquinone/ubiquinol; (b) a surfactant which is a member selected from PTS, PSS, PCS, and PQS. In an exemplary embodiment, the formulation further comprises rice bran oil. In an exemplary embodiment, the formulation further comprises beeswax. In an exemplary embodiment, the formulation further comprises carotenoids.

In another aspect, the formulation comprises (a) a ubiquinone/ubiquinol; (b) a surfactant which is a member selected from PTS, PSS, PCS, and PQS; rice bran oil; beeswax; and carotenoids. In another exemplary embodiment, the formulation comprises (a) a ubiquinone/ubiquinol; (b) PTS; omega-3 enriched fish oil; beeswax; and carotenoids.

### Methods of Making the Formulations

In another aspect, the invention provides a method of making the formulations described herein.

In an exemplary embodiment, the method of making the formulation comprises (i) contacting said ubiquinone/ubiquinol and said surfactant; and (ii) contacting the product of step (i) with said lipophilic carrier and said viscosity enhancer, thereby making the formulation.

In another exemplary embodiment, the method of making the formulation comprises (i) melting the beeswax in the lipophilic carrier and heating the resulting mixture to a minimum of 50° C. until the wax has melted completely and the solution is clear. The method may further include any of the following steps: (ii) cooling the mixture to at least 30° C.; (iii) adding the viscosity enhancer; (iv) mixing the intermediate mixture for at least 20 minutes; (v) adding the ubiquinone/ubiquinol, preferably at a temperature of 28° C. or less; and mixing the product of step (v) for a minimum of 30 minutes to form the formulation.

In another exemplary embodiment, the method of making the formulation comprises: (i) heating the lipophilic carrier to 50 to 60° C. (ii) adding beeswax (50° C. is above the melting point of bees wax); (iii) mixing the wax and lipophilic carrier until a uniform mixture is formed. Bees wax thickens the lipophilic carrier and acts as a suspension agent for subsequent ingredients. Without bees wax, the other ingredients, when suspended inside a transparent gel capsule, might separate or congregate under the effect of gravity, and appear faulty or spoiled to the consumer. The method may further include (iv) cooling the mixture of step (iii) to 35 to 45° C.; (v) adding ubiquinone/ubiquinol under a vacuum (to eliminate oxidation) and mixing the resulting mixture for one to two hours; (vi) cooling the resultant mixture to 25 to 30° C. A nitrogen gas blanket is introduced to shield the mixture for oxygen and the pressure is returned to atmospheric. The mixture is then encapsulated in a soft gel capsule.

In another exemplary embodiment, the method of making the formulation comprises: (i) Mixing all ingredients under a nitrogen blanket and maintain this blanket throughout blending; (ii) Melting the viscosity enhancer as well as other components in the lipophilic carrier, and heating the mixture to a minimum of 60° C.; (iii) Allowing the mixture to cool to at least 26° C. and adding CoQ₁₀; (iv) Mixing the solution for a minimum of 30 minutes to assure the mixture is homogenous and that no air remains; and (v) Encapsulate in a gel capsule.

In an exemplary embodiment, the invention provides a first formulation comprising: a) a ubiquinone/ubiquinol; b) a surfactant; c) a lipophilic carrier; and d) a viscosity enhancer wherein said surfactant is a member selected from polyoxyethanyl-tocopheryl-sebacate (PTS), polyoxyethanyl-sitosterol-sebacate (PSS), polyoxyethanyl-cholesterol-sebacate (PCS), polyoxyethanyl-ubiquinol-sebacate (PQS) and combinations thereof.

In an exemplary embodiment, a second formulation, wherein the first formulation described herein, is encompassed within a soft gel capsule. In an exemplary embodiment, wherein said ubiquinone/ubiquinol is CoQ₁₀. In an exemplary embodiment, wherein said lipophilic carrier is a member selected from an essential oil, an essential oil component, fish oil, vegetable oil, rice bran oil, soybean oil, an oil comprising an omega-3 fatty acid, an oil comprising an omega-6 fatty acid, and oil comprising an omega-9 fatty acid, and combinations thereof. In an exemplary embodiment, wherein said lipophilic carrier is an oil comprising an omega-3 fatty acid, and said omega-3 fatty acid is alpha-linolenic acid. In an exemplary embodiment, wherein said lipophilic carrier is an oil comprising an omega-6 fatty acid, and said omega-6 fatty acid is a member selected from linoleic acid and arachidonic acid. In an exemplary embodiment, wherein said lipophilic carrier is an oil comprising an omega-9 fatty acid, and said omega-9 fatty acid is oleic acid. In an exemplary embodiment, wherein said viscosity enhancer is beeswax. In an exemplary embodiment, wherein the ratio of said ubiquinone/ubiquinol to said surfactant is from about 1:1 to about 1:5. In an exemplary embodiment, wherein the ratio of said ubiquinone/ubiquinol to said surfactant is from about 1:1 to about 1:3. In an exemplary embodiment, wherein the ratio of said ubiquinone/ubiquinol to said surfactant is from about 1:1 to about 1:2. In an exemplary embodiment, wherein the ratio of said ubiquinone/ubiquinol to said surfactant is about 1:1.5. In an exemplary embodiment, wherein said capsule is essentially free of precipitation within said capsule. In an exemplary embodiment, wherein said capsule is essentially free of precipitated ubiquinone/ubiquinol within said capsule. In an exemplary embodiment, wherein the average particle size of said formulation in an aqueous solution or an aqueous-based solution is from about 10 nm to about 90 nm. In an exemplary embodiment, wherein the average particle size of said formulation in an aqueous solution or an aqueous-based solution is from about 10 nm to about 30 nm. In an exemplary embodiment, wherein the average particle size of said formulation in an aqueous solution or an aqueous-based solution is from about 20 nm to about 30 nm. In an exemplary embodiment, wherein said surfactant is PTS, said lipophilic carrier is omega fatty acid enriched fish oil, and said viscosity enhancer is beeswax. In an exemplary embodiment, wherein said formulation is encapsulated within a soft gel capsule. In an exemplary embodiment, wherein the ratio of said ubiquinone/ubiquinol to said PTS is from about 1:1 to about 1:2. In an exemplary embodiment, further comprising a compound which is a member selected from a pharmaceutical drug molecule, a sterol, a vitamin, a provitamin, an amino acid, an amino acid analog, a fat, a phospholipid, a carotenoid, a sugar, a starch, an antibiotic, an antioxidant, a reducing agent, an anti-oxidant and a free radical scavenger. In an exemplary embodiment, further comprising a pharmaceutically acceptable additive. In an exemplary embodiment, wherein said additive is a member selected from a solvent, emulsifier, adjuvant, sweetener, filler, colorant, flavoring agent, lubricant, binder, moisturizing agent, preservative, mineral, suspending agent and mixtures thereof. In an exemplary embodiment, the invention provides a method of making the formulation described herein. In an exemplary embodiment, the invention provides a method of making the formulation, comprising: (i) contacting said bioactive lipophilic molecule and said surfactant, and (ii) contacting the product of step (i) with said lipophilic carrier and said viscosity enhancer, thereby making the formulation. In an exemplary embodiment, the bioactive lipophilic molecule is CoQ10. In another aspect, the invention provides a formulation comprising: a) a bioactive lipophilic molecule which is a member selected from asiatic acid, ursolic acid, lutein, astaxanthin, curcumins, beta-carotene, lycopene, resveratrol, lecithin, L-carnitine (or acetyl derivative), tocotrienols, alpha-lipoic acid, salmon oil, grape seed extract, bilberry extract, flaxseed oil, garlic oil, ginkgo biloba extract, pumpkin seed oil, green tea catechins extract, kava, evening primrose oil, wheat germ oil, hyaluronic acid, saw palmetto berry oil extract, ginseng, Japanese knotwood extract, phytosterols, hawthorne, St. John's wort, melatonin, valerian yohimbe, ephedra, red clover, cayenne, echinacea, arnica Montana, docosahexaenoic acid and combinations thereof, b) a surfactant; c) a lipophilic carrier; wherein said surfactant is a member selected from polyoxyethanyl-tocopheryl-sebacate (PTS), polyoxyethanyl-sitosterol-sebacate (PSS), polyoxyethanyl-cholesterol-sebacate (PCS), polyoxyethanyl-ubiquinol-sebacate (PQS) and combinations thereof. In an exemplary embodiment, further comprising d) a viscosity enhancer.

The invention is further illustrated by the Examples that follow. The Examples are not intended to define or limit the scope of the invention.

### EXAMPLES

The following abbreviations are used throughout the Examples:

CoQ₁₀—coenzyme Q₁₀

Ub50—ubiquinol-50

PQS—polyoxyethanyl-ubiquinol-sebacate

PTS—polyoxyethanyl-tocopherol-sebacate

A number following one of the above abbreviations (e.g., PQS-600) indicates an average molecular weight of the polyoxyethanyl moiety of the compound. A number followed by Me abbreviation (e.g., PQS-750Me) indicates a polyoxyethanyl moiety capped with a methyl group (methoxypolyoxyethanyl).

### Example 1

### Exemplary Formulations of CoQ₁₀

Formulations were prepared which contained the following amounts of CoQ₁₀, PTS, Oil (either rice bran oil or omega-fatty acid enriched fish oil (ONC Oil 18/12)) and beeswax:

| | Components [mg] | | | | | | | |
|---|---|---|---|---|---|---|---|---|
| | A | B | C | D | E | F | G | H |
| CoQ₁₀ | 100 | 100 | 100 | 100 | 100 | 100 | 100 | 100 |
| PTS | 150 | 150 | 150 | 150 | 150 | 150 | 150 | 150 |
| ONC Oil 18/12 | 300 | — | 300 | — | 200 | — | 200 | — |
| Rice Bran Oil | — | 300 | — | 300 | — | 200 | — | 200 |
| Beeswax | 50 | 50 | 25 | 25 | 25 | 25 | 50 | 50 |
| Total weight | 600 | 600 | 575 | 575 | 475 | 475 | 500 | 500 |
| (mg) | | | | | | | | |

These formulations were produced on the following dates:
Formulation A: Sep. 19, 2006
Formulation B: Sep. 24, 2006
Formulations C- H: Oct. 10, 2006
   The formulations were kept at room temperature under argon since the date of production to test for stability and formation of precipitate. No precipitate was noted by visual observation on Nov. 22, 2006.

Formulations A-H were prepared by melting CoQ₁₀ and PTS and mixing them together at 60° C. To the cooled formulations were added oil and beeswax. The formulations were incubated at 60° C. until the beeswax melted. The formulations were then mixed again and sealed under argon gas.

### Example 2

### Particle Size Distribution of Micelles

Formulations of PTS and CoQ₁₀ (3:1) as well as PTS alone were diluted with water (8×, 16×, and 32× mg/mL). Formulations had a concentration of 1-5 mg PTS/mL water. The resulting solutions were filtered through a filter cartridge to remove dust particles, transferred to a glass sample tube and placed into an oil bath for temperature control. The particle size distribution of the formed micelles in the formulations were subsequently measured at 25° C. using a Brookhaven Dynamic Light Scattering (DLS) detector equipped with an Avalanche photodiode detector and an MG vertically polarized 35 mmV Helium-Neon 633 nm laser.

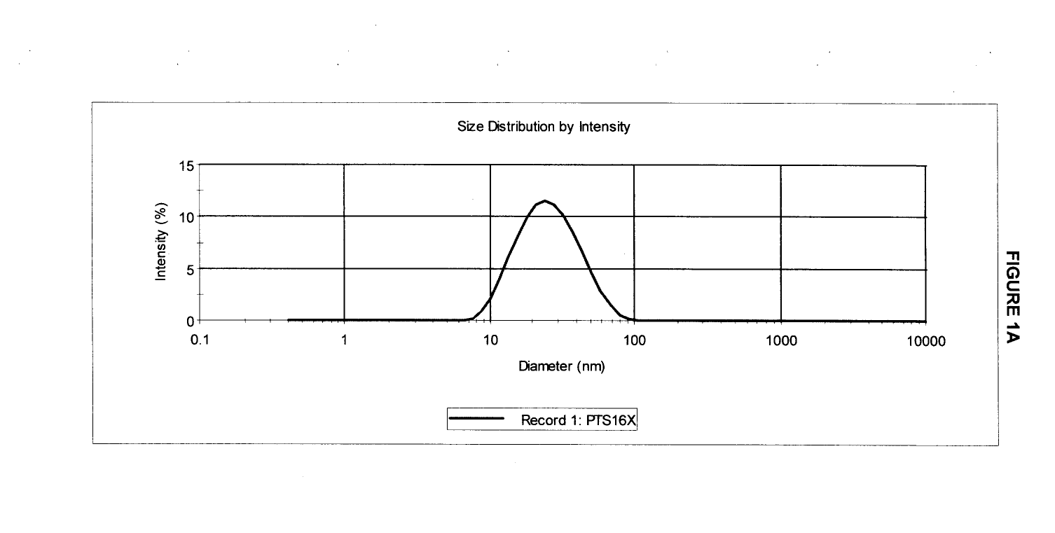

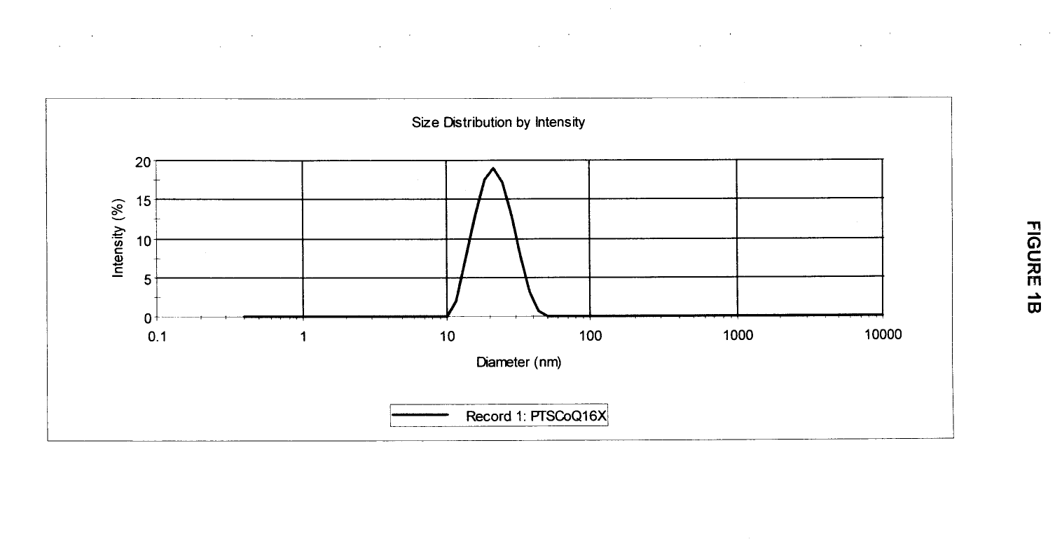

The obtained particle size distribution curves showed that the particle sizes for each of the tested CoQ10/PTS formulations are generally smaller than 60 nm and the median particle size is about 22 nm. Similar values were obtained for PTS alone. However, the size distribution for PTS alone was found to be broader. Representative particle size-distribution curves comparing 16× dilutions of PTS and a 3:1 composition of PTS/CoQ₁₀ are shown in FIG. 1A and FIG. 1B, respectively.

### Example 3

### Preparation of Polyoxyethanyl-sitosteryl Sebacate (PSS-600)

0.83 g of b-sitosterol (Sigma Chem. Co., product #S-5753, approximately 60%) was dissolved in 3 mL of dry toluene at 40° C., followed by addition of 1.33 mmole of triethylamine (TEA). 1.33 mmole of sebacoyl chloride dissolved in 2 mL of dry toluene was than added (dropwise, while stirring, and under anhydrous conditions) to the b-sitosterol-TEA solution. The reaction was carried out for 10 min at room temperature, at which time 2 mmole of PEG-600 (polyethylene glycol, Sigma Chem. Co., product # P-3390) and 2.66 mmole of TEA dissolved in 3 mL of dry toluene were added dropwise to the reaction mixture. The reaction was continued with stirring for additional 20 min at room temperature and the reaction mixture was extracted four times with 3 mL each time of saturated solution of NaCl. The toluene was removed under reduced pressure leaving a waxy residue. This product was dissolved in 15 mL of water and water-insoluble materials removed by filtration. The filtrate was lyophilized, yielding 0.8 g of pale-yellow waxy product (PSS-600). The same method was used for the preparation of polyoxyethanyl-cholesteryl sebacate (PCS-600).

### Example 4

### Preparation of Polyoxyethanyl-a-Tocopheryl Sebacate (PTS-600)

A solution of 1 mmole of a -tocopherol (Sigma Chem. Co., product # T-3251) and 1.33 mmole of TEA in 3 mL of dry toluene was added (dropwise, under anhydrous conditions, while stirring) to 1.33 mmole of sebacoyl chloride dissolved in 2 mL of dry toluene. The reaction was carried out for 10 min at room temperature, followed by a dropwise addition of 2 mmole of PEG-600 (polyethylene glycol, Sigma, P-3390) and 2.66 mmole of TEA dissolved in 3 mL of toluene. The reaction was continued for additional 20 min at room temperature with constant stirring. The reaction mixture was extracted four times with 3 mL each time of saturated solution of NaCl and toluene evaporated under a reduced pressure. The product was dissolved in 5 mL of water and the residual toluene was further removed by co-evaporation with water under a reduced pressure. The final waxy product (1.15 g) was obtained by lyophilization.

Other solubilizing agents can be obtained by linking polyethylene glycol (average molecular weight 1000, Sigma Chem. Co., product # P-3515) or methoxypolyethylene glycol (average molecular weight 750, Sigma Chem. Co., product # M-7018) to a-tocopherol using adipoyl, suberoyl, azelaoyl or dodecanedioyl dichlorides. They can be synthesized according to the method of Example 4.

### Example 4

### A Method of Determining Precipitation in a Second Formulation

A 50 mg sample of the contents of a soft gel was dissolved/dispersed in 0.5 mL water by shaking for 1 min. The resulting suspension was placed in a water bath at 37° C. The sample was then centrifuged for 1 min. In cases where precipitation was observed, the pale yellow supernatant was removed leaving behind a pellet of pure coenzyme Q10. Solid beeswax was not formed (which would settle on top of the supernatant). With the current formulation, <5% CoQ10 precipitation was observed by isolation and microscopic examination of the solid material.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference for all purposes.

What is claimed is:

1. A first formulation comprising:
a) a ubiquinone/ubiquinol;
b) a surfactant;
c) a lipophilic carrier; and
d) a viscosity enhancer
wherein said surfactant is a member selected from polyoxyethanyl-tocopheryl-sebacate (PTS), polyoxyethanyl-sitosterol-sebacate (PSS), polyoxyethanyl-cholesterol-sebacate (PCS), polyoxyethanyl-ubiquinol-sebacate (PQS) and combinations thereof.

2. A second formulation, wherein the first formulation of claim 1, is encompassed within a soft gel capsule.

3. The first formulation of claim 1, wherein said ubiquinone/ubiquinol is CoQ₁₀.

4. The first formulation of claim 1, wherein said lipophilic carrier is a member selected from an essential oil, an essential oil component, fish oil, vegetable oil, rice bran oil, soybean oil, an oil comprising an omega-3 fatty acid, an oil comprising an omega-6 fatty acid, and oil comprising an omega-9 fatty acid, and combinations thereof.

5. The first formulation of claim 4, wherein said lipophilic carrier is an oil comprising an omega-3 fatty acid, and said omega-3 fatty acid is alpha-linolenic acid.

6. The first formulation of claim 4, wherein said lipophilic carrier is an oil comprising an omega-6 fatty acid, and said omega-6 fatty acid is a member selected from linoleic acid and arachidonic acid.

7. The first formulation of claim 4, wherein said lipophilic carrier is an oil comprising an omega-9 fatty acid, and said omega-9 fatty acid is oleic acid.

8. The first formulation of claim 1, wherein said viscosity enhancer is beeswax.

9. The first formulation of claim 1, wherein the ratio of said ubiquinone/ubiquinol to said surfactant is from about 1:1 to about 1:5.

10. The first formulation of claim 1, wherein the ratio of said ubiquinone/ubiquinol to said surfactant is from about 1:1 to about 1:3.

11. The first formulation of claim 1, wherein the ratio of said ubiquinone/ubiquinol to said surfactant is from about 1:1 to about 1:2.

12. The first formulation of claim 1, wherein the ratio of said ubiquinone/ubiquinol to said surfactant is about 1:1.5.

13. The first formulation of claim 2, wherein said capsule is essentially free of precipitation within said capsule.

14. The formulation of claim 2, wherein said capsule is essentially free of precipitated ubiquinone/ubiquinol within said capsule.

15. The formulation of claim 1, wherein the average particle size of said formulation in an aqueous solution or an aqueous-based solution is from about 10 nm to about 90 nm.

16. The formulation of claim 15, wherein the average particle size of said formulation in an aqueous solution or an aqueous-based solution is from about 10 nm to about 30 nm.

17. The formulation of claim 16, wherein the average particle size of said formulation in an aqueous solution or an aqueous-based solution is from about 20 nm to about 30 nm.

18. The formulation of claim 3, wherein said surfactant is PTS, said lipophilic carrier is omega fatty acid enriched fish oil, and said viscosity enhancer is beeswax.

19. The formulation of claim 18, wherein said formulation is encapsulated within a soft gel capsule.

20. The formulation of claim 19, wherein the ratio of said ubiquinone/ubiquinol to said PTS is from about 1:1 to about 1:2.

21. The formulation of claim 1, further comprising a compound which is a member selected from a pharmaceutical drug molecule, a sterol, a vitamin, a provitamin, an amino acid, an amino acid analog, a fat, a phospholipid, a carotenoid, a sugar, a starch, an antibiotic, an antioxidant, a reducing agent, an anti-oxidant and a free radical scavenger.

22. The formulation of claim 1, further comprising a pharmaceutically acceptable additive.

23. The formulation of claim 22, wherein said additive is a member selected from a solvent, emulsifier, adjuvant, sweetener, filler, colorant, flavoring agent, lubricant, binder, moisturizing agent, preservative, mineral, suspending agent and mixtures thereof.

24. A method of making the formulation of claim 1.

25. The method of making the formulation of claim 1, comprising:
(i) contacting said ubiquinone/ubiquinol and said surfactant
(ii) contacting the product of step (i) with said lipophilic carrier and said viscosity enhancer, thereby making the formulation.

26. A formulation comprising:
a) a bioactive lipophilic molecule which is a member selected from asiatic acid, ursolic acid, lutein, astaxanthin, curcumins, beta-carotene, lycopene, resveratrol, lecithin, L-carnitine (or acetyl derivative), tocotrienols, alpha-lipoic acid, salmon oil, grape seed extract, bilberry extract, flaxseed oil, garlic oil, ginkgo biloba extract, pumpkin seed oil, green tea catechins extract, kava, evening primrose oil, wheat germ oil, hyaluronic acid, saw palmetto berry oil extract, ginseng, Japanese knotwood extract, phytosterols, hawthorne, St. John's wort, melatonin, valerian yohimbe, ephedra, red clover, cayenne, echinacea, arnica Montana, docosahexaenoic acid and combinations thereof;
b) a surfactant;
c) a lipophilic carrier; and
wherein said surfactant is a member selected from polyoxyethanyl-tocopheryl-sebacate (PTS), polyoxyethanyl-sitosterol-sebacate (PSS), polyoxyethanyl-cholesterol-sebacate (PCS), polyoxyethanyl-ubiquinol-sebacate (PQS) and combinations thereof.

27. The formulation of claim 26, further comprising
d) a viscosity enhancer.
